(19) United States
(12) Patent Application Publication
(10) Pub. No.: US 2004/0149293 A1
(43) Pub. Date: Aug. 5, 2004

(54) Pediatric arm restraint

(76) Inventor: Shannon L. Freedman, Newport Coast, CA (US)

Correspondence Address: Schwartz Law Firm, P.C., SouthPark Towers, Suite 530, 6100 Fairview Road, Charlotte, NC, 28210, US

(21) Appl. No.: 10/751,757

(22) Filed: Jan. 5, 2004

Related U.S. Application Data
(60) Provisional application No. 60/437,843, filed on Jan. 3, 2003.

(51) Int. Cl. A61F 5/37

(52) U.S. Cl. 128/878

(57) ABSTRACT

An arm restraint is adapted for limiting arm movement of a patient. The arm restraint includes a fabric arm wrap designed for receiving and folding around an arm of the patient in a generally extended condition. The arm wrap has a first end adapted for residing adjacent an upper arm of the patient and a second end residing adjacent a wrist of the patient. A plurality of spaced-apart elongated stays are incorporated within the arm wrap, and extend longitudinally from its first end to its second end. The stays cooperate to restrict bending of the arm at the elbow. A fabric shoulder fold is formed with the first end of the arm wrap, and is designed for extending over and covering a shoulder of the patient adjacent the upper arm. A torso strap is secured to the shoulder fold at a first attachment point. The torso strap has a free end designed for extending across and around an upper torso of the patient. The free end attaches to the shoulder fold at a second attachment point. The torso strap operates to secure and maintain the arm restraint in position on the arm of the patient.

### TECHNICAL FIELD AND BACKGROUND OF INVENTION

This application relates to a pediatric arm restraint. The invention is especially applicable for limiting arm movement of a young child to prevent the child from disturbing sutures and irritating an incision following cranio-facial surgery. The invention is worn on each arm of the child, and operates to restrict bending of the arm at the elbow.

According to the American Cleft Palate Association, there are approximately 5,000 cases per year of cleft palate and cleft lip. Many cases involve two or more corrective surgeries. These numbers align with government data which indicates the frequency of children born with cleft palate to be 1 in 1000 cases (0.1%). According to the U.S. Census Bureau, the annual number of births is slightly over 4 million. Therefore, there are approximately 4,000 surgeries per year for cleft palate alone. Additional reasons for cranio-facial surgery include ear deformities, premature fusing of skull bones, misshapen jaws, and facial asymmetries. Using market estimates, the number of pediatric arm immobilizers or “splints” used domestically is approximately 20,000 pairs per year.

Wrap-around arm splints especially designed for children are well known in the prior art. According to one commercially available product, splints are worn on each arm of the child and are interconnected using an adjustable strap held tight across the back between the two splints. The strap attaches to each of the splints, and holds the splints in place on the arms by leveraging one splint off of the another. Unfortunately, when a child is uncomfortable after an operation, he or she generally wiggles and arches the back which effectively loosens the strap, thereby allowing the strap and the arm splints to slip downward out of position. To account for this, the splints are frequently pulled up as high as possible on the arm, pushing uncomfortably into the armpit of the child.

In addition, the prior art splint is secured on the arm using a single large strip of Velcro® hook-and-loop fastener running the length of the splint. Opening the splint is very loud and requires pressing against/pulling the arm in order to attach and adjust the Velcro®, both of which greatly disturb the child and discourage efforts to keep the child calm and comfortable after surgery. A further disadvantage of this fastener design is that the entire arm must be released even if the splint requires tightening in only one particular area. In adjusting the splint on the one arm, the splint on the other arm is often inadvertently released—as the strap across the back is difficult to keep tight during adjustment. This design is also difficult to tighten around the arm, resulting in frequently disturbing the child for additional adjustments. Because the two arm splints are interconnected using a single strap, when adjusting the splints, changing clothes, or exercising the child's arms, it is generally necessary to have two people present in order to release and control both arms of the child. Releasing both arms further increases the chances of the child disturbing the suture.

The present invention addresses these and other disadvantages of the prior art, as discussed below.

### SUMMARY OF INVENTION

Therefore, it is an object of the invention to provide an arm restraint which is especially designed for a young child.

It is another object of the invention to provide an arm restraint which is easy-to-use and comfortable, and that remains properly positioned on the arm for long periods of time without the need for frequent readjustment.

It is another object of the invention to provide an arm restraint which is relatively light, and provides even pressure along the entire length of the arm.

It is another object of the invention to provide an arm restraint which is launderable.

It is another object of the invention to provide an arm restraint which uses multiple wrap-around straps for easier application, adjustment, and removal.

It is another object of the invention to provide an arm restraint which has a generally “arm-sleeve”, shape for a better, more comfortable fit.

It is another object of the invention to provide an arm restraint which utilizes a separate torso strap to independently retain the device on the arm. This allows the arms to be more easily controlled when exercising, changing clothes, or adjusting the restraint. Using the present invention, one arm of the child can be released and exercised and then placed back in the restraint easily by just one parent, while the other restraint is left in place to restrict bending of the other arm.

It is another object of the invention to provide an arm restraint which includes a soft, thin, felt-like or quilted material against the skin to substantially eliminate any discomfort or abrasion caused by the restraint.

It is another object of the invention to provide an arm restraint which has a relatively short length extending only to a point above the wrist thereby allowing full range of hand movement.

It is another object of the invention to allow separate and independent adjustment of both right and left arm restraints.

It is another object of the invention to provide an arm restraint which includes multiple independent adjustment points.

It is another object of the invention to provide an arm restraint which can be applied, adjusted, and removed without disturbing the child.

It is another object of the invention to provide an arm restraint which is formed in multiple lengths to accommodate children of different size and age.

It is another object of the invention to provide an arm restraint which allows the child to play freely and comfortably.

It is another object of the invention to provide an arm restraint which utilizes a quilted, soft, and colorful material.

It is another object of the invention to provide an arm restraint which is both comfortable for the child and aesthetically pleasing to the parents.

It is another object of the invention to provide an arm restraint which is not bulky and relatively easy to hide under clothing.

It is another object of the invention to provide an arm restraint which is specifically designed to fit comfortably high on the shoulder and low under the arm-pit of the child.

These and other objects of the present invention are achieved in the preferred embodiments disclosed below by providing an arm restraint adapted for limiting arm movement of a patient, such as a young child. The arm restraint includes a fabric arm wrap designed for receiving and folding around an arm of the patient in a generally extended condition. The arm wrap has a first end adapted for residing adjacent an upper arm of the patient and a second end residing adjacent a wrist of the patient. A plurality of spaced-apart elongated stays are incorporated within the arm wrap, and extend longitudinally from its first end to its second end. The stays cooperate to restrict bending of the arm at the elbow. A fabric shoulder fold is formed with the first end of the arm wrap, and is designed for extending over and covering a shoulder of the patient adjacent the upper arm. A torso strap is secured to the shoulder fold at a first attachment point. The torso strap has a free end designed for extending across and around an upper torso of the patient. The free end attaches to the shoulder fold at a second attachment point. The torso strap operates to secure and maintain the arm restraint in position on the arm of the patient.

According to another preferred embodiment, means are provided for adjusting the fit of the arm wrap around the arm of the patient.

According to another preferred embodiment, the means for adjusting includes complementary hook and loop fasteners.

Preferably, the means for adjusting includes a plurality of longitudinally spaced wrap-around adjustment straps.

Preferably, the plurality of wrap-around adjustment straps includes a wrist strap, an elbow strap, and an upper arm strap.

According to another preferred embodiment, each of the adjustment straps includes complementary hook and look fasteners.

According to another preferred embodiment, a fabric liner is attached to an inside of the arm wrap and provides a comfort barrier against the skin of the patient.

Preferably, the fabric liner is formed of an anti-microbial material.

Preferably, the arm wrap is formed of a flame retardant fabric.

According to another preferred embodiment, the torso strap includes elastic yarns.

According to another preferred embodiment, the second attachment point at the shoulder fold includes a grommet adapted for receiving the free end of the torso strap.

According to another preferred embodiment, a lateral hinge line is located between the shoulder fold and the arm wrap.

According to another preferred embodiment, a neck fold is formed with the shoulder fold and adapted for residing adjacent a neck of the patient.

According to another preferred embodiment, a lateral hinge line is located between the neck fold and the shoulder fold.

### BRIEF DESCRIPTION OF THE DRAWINGS

Some of the objects of the invention have been set forth above. Other objects and advantages of the invention will appear as the description proceeds when taken in conjunction with the following drawing, in which:

FIG. 1 is a perspective of the pediatric arm restraint according to one preferred embodiment of the invention;

FIG. 2 is a side view of the pediatric arm restraint in an opened, unfolded condition;

FIG. 3 is an opposite side view of the pediatric arm restraint in the opened condition;

FIG. 4 is a perspective view of the pediatric arm restraint applied to the arm of a child; and

FIG. 5 is a further perspective view of the pediatric arm restraint applied to the arm of the child.

### DESCRIPTION OF THE PREFERRED EMBODIMENT AND BEST MODE

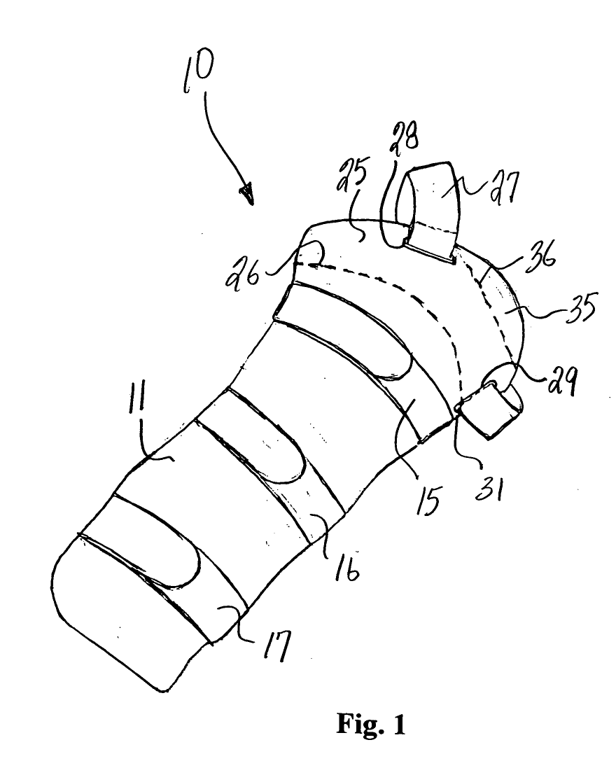

Referring now specifically to the drawings, a pediatric arm restraint according to the present invention is illustrated in FIG. 1, and shown generally at reference numeral 10. The arm restraint 10 is intended to be worn on each arm to prevent the child from disturbing delicate sutures after cranio-facial surgery. Unlike prior art devices discussed above, the two arm restraints 10 are secured to the child independent of one another, are comfortable, allow free movement of the hands and wrists, and are conveniently applied to and removed from the child with minimal disturbance.

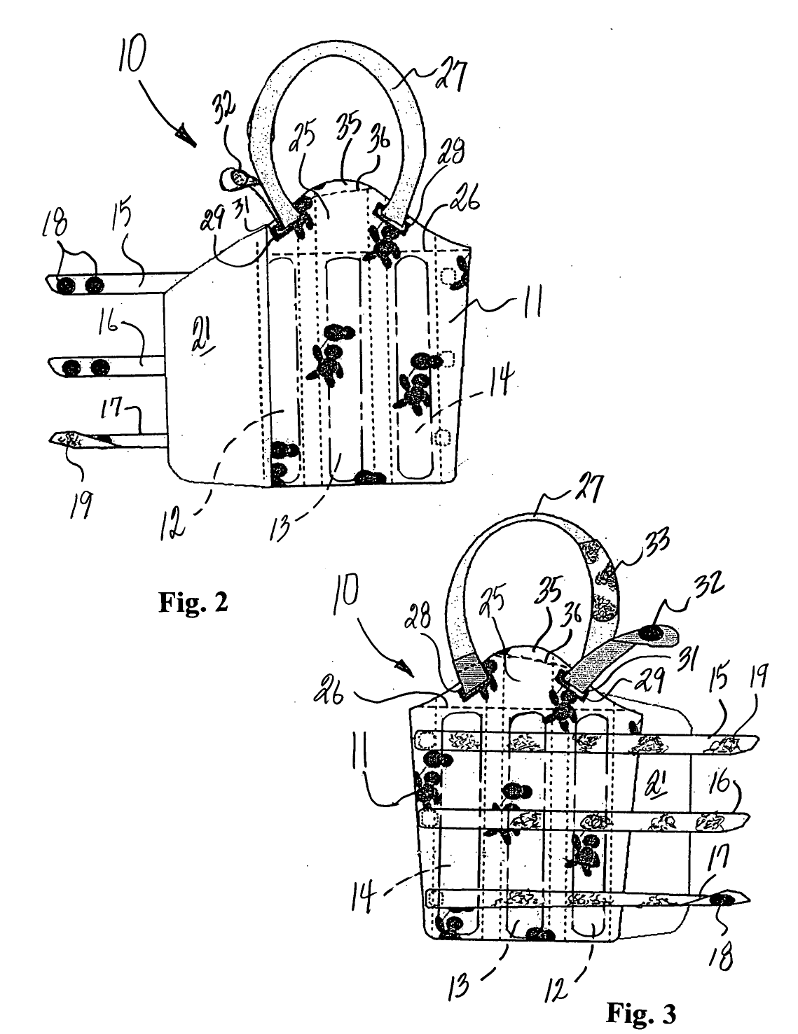

As best shown in FIGS. 2 and 3, the arm restraint 10 comprises a quilted fabric arm wrap 11 with three sewn-in longitudinal stays 12, 13 and 14. The stays 12, 13, and 14 extend from one end of the arm wrap 11 to the other, and ar formed of a rigid or semi-rigid plastic. Wrap-around adjustment straps 15, 16, and 17 are sewn to the outside of the arm wrap 11, and serve to custom fit and secure the arm restraint 10 to the arm of the child. Opposing major surfaces of each strap 15-17 include complementary hook and loop fasteners 18 and 19, such as Velcro®. The hook fasteners 18 of each strap 15-17 are preferably formed in two small patches which create little noise when released from the loop fasteners 19, and which require relatively little pulling to separate from the loop fasteners 19. The loop fasteners 19 preferably extend the entire length of the straps 15-17. According to the embodiment shown, the adjustment straps 15-17 are located generally at the wrist, elbow and upper arm, respectively. When properly worn by the child, the adjustment straps 15-17 and longitudinal stays 12-14 of the arm restraint 10 cooperate to prevent bending of the arm at the elbow. For added comfort, a relatively thin fabric liner 21 is sewn to the inside of the fabric wrap 11 and resides between the skin of the child and adjustment straps 15-17. The liner 21 is preferably formed of a soft, breathable, anti-microbial fabric.

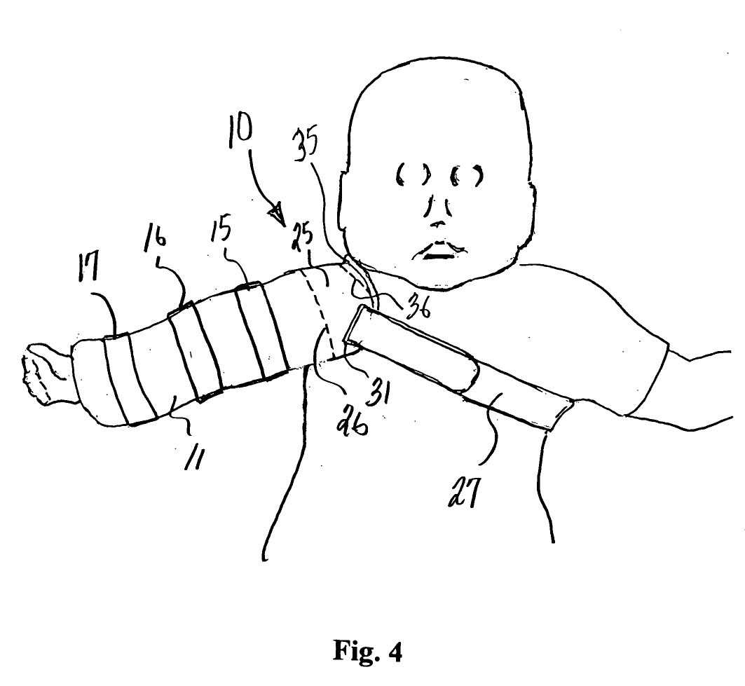

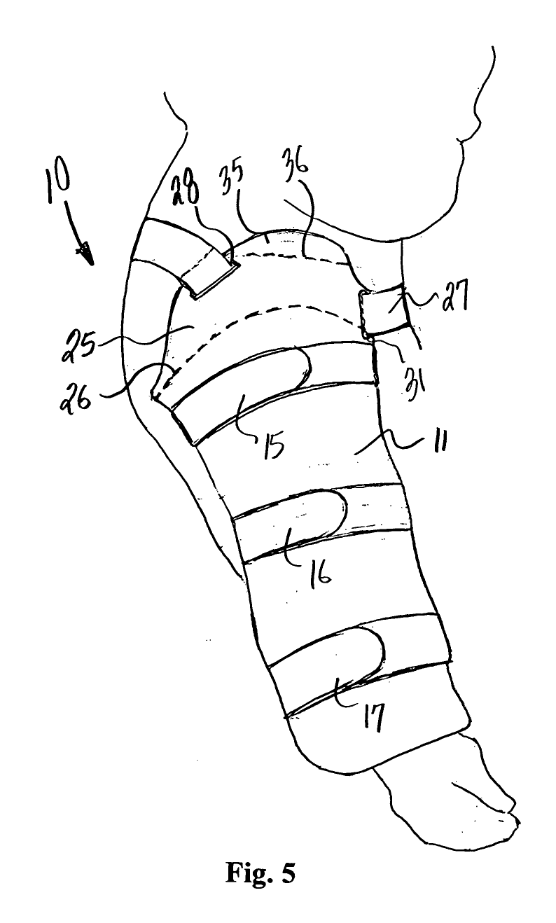

A fabric shoulder fold 25 is formed with the end of the arm wrap 11 along a lateral hinge line 26. As best shown in FIGS. 4 and 5, the shoulder fold 25 is designed to extend over and cover the shoulder of the child adjacent the upper arm. The hinge line 26 is located between the shoulder fold 25 and arm wrap 11, and facilitates proper and secure placement of the arm restraint 10 on the shoulder and arm of the child without sacrificing the child's ability to lift and lower the arm. An elastic torso strap 27 is attached to the shoulder fold 25 at a first attachment point 28, and has a free end designed to extend across and around the upper torso of the child passing under the opposite arm. The free end passes through a grommet 29 located at a second attachment point 31 on the shoulder fold 25, and is doubled over to mate complementary patches of hook and loop fasteners 32 and 33. The second attachment point 31 is located a sufficient distance from the first point 28 to move the strap 27 safely away from the child's neck. Once attached, the torso strap 27 pulls the arm wrap 11 towards the body from above shoulder of the child. This attachment leverage prevents the arm wrap 11 from slipping downward along the length of the arm, and retains the arm restraint 10 in proper effective position as the child lifts and moves the arm. Because the arm wrap 11 extends only to a point above the wrist, the child is free to use his or her hand for holding toys, comfort objects, and loved ones. This feature is especially important given that most children generally recover from surgery within a couple of days, while the sutures may require two or more weeks to properly secure the incision.

For still further comfort, a neck fold 35 is preferably formed with the shoulder fold 25 along a second lateral hinge line 36. The neck fold 35 resides at the side of the child's neck, and bends upwardly to cushion against the skin as the child lifts and moves the arm. Each of the hinge lines 26 and 36 are preferably formed by sewing stitches.

A pediatric arm restraint is described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

I claim:

1. An arm restraint adapted for limiting arm movement of a patient, said restraint comprising:
(a) a fabric arm wrap designed for receiving and folding around an arm of the patient in a generally extended condition, said arm wrap having a first end adapted for residing adjacent an upper arm of the patient and a second end residing adjacent a wrist of the patient;
(b) a plurality of spaced-apart elongated stays incorporated within said arm wrap, and extending longitudinally from the first end thereof to the second end thereof, said stays cooperating to restrict bending of the arm at the elbow;
(c) a fabric shoulder fold formed with the first end of said arm wrap and designed for extending over and covering a shoulder of the patient adjacent the upper arm; and
(d) a torso strap secured to said shoulder fold at a first attachment point, and having a free end designed for extending around an upper torso of the patient and attaching to said shoulder fold at a second attachment point, said torso strap operating to secure and maintain the arm restraint in position on the arm of the patient.

2. An arm restraint according to claim 1, and comprising means for adjusting the fit of said arm wrap around the arm of the patient.

3. An arm restraint according to claim 2, wherein said means for adjusting comprises complementary hook and loop fasteners.

4. An arm restraint according to claim 2, wherein said means for adjusting comprises a plurality of longitudinally spaced wrap-around adjustment straps.

5. An arm restraint according to claim 4, wherein said plurality of wrap-around adjustment straps comprises a wrist strap, an elbow strap, and an upper arm strap.

6. An arm restraint according to claim 5, wherein each of said adjustment straps comprises complementary hook and look fasteners.

7. An arm restraint according to claim 1, and comprising a fabric liner attached to an inside of said arm wrap and providing a comfort barrier against the skin of the patient.

8. An arm restraint according to claim 7, wherein said fabric liner comprises an anti-microbial material.

9. An arm restraint according to claim 1, wherein said arm wrap comprises a flame retardant fabric.

10. An arm restraint according to claim 1, wherein said torso strap comprises elastic yarns.

11. An arm restraint according to claim 1, wherein the second attachment point at said shoulder fold comprises a grommet adapted for receiving the free end of said torso strap.

12. An arm restraint according to claim 1, and comprising a lateral hinge line between said shoulder fold and said arm wrap.

13. An arm restraint according to claim 1, and comprising a neck fold formed with said shoulder fold and adapted for residing adjacent a neck of the patient.

14. An arm restraint according to claim 1, and comprising a lateral hinge line between said neck fold and said shoulder fold.

15. An arm restraint adapted for limiting arm movement of a patient, said restraint comprising:
(a) a fabric arm wrap designed for receiving and folding around an arm of the patient in a generally extended condition, said arm wrap having a first end adapted for residing adjacent an upper arm of the patient and a second end residing adjacent a wrist of the patient;
(b) means for adjusting the fit of said arm wrap around the arm of the patient.
(c) a plurality of spaced-apart elongated stays incorporated within said arm wrap, and extending longitudinally from the first end thereof to the second end thereof, said stays cooperating to restrict bending of the arm at the elbow;
(d) a fabric liner attached to an inside of said arm wrap and providing a comfort barrier against the skin of the patient;
(e) a fabric shoulder fold formed with the first end of said arm wrap and designed for extending over and covering a shoulder of the patient adjacent the upper arm; and
(f) a torso strap secured to said shoulder fold at a first attachment point, and having a free end designed for extending around an upper torso of the patient and attaching to said shoulder fold at a second attachment point, said torso strap operating to secure and maintain the arm restraint in position on the arm of the patient.

16. An arm restraint according to claim 15, wherein said means for adjusting comprises a plurality of longitudinally spaced wrap-around adjustment straps, said adjustment straps comprising a wrist strap, an elbow strap, and an upper arm strap.

17. An arm restraint according to claim 15, and comprising a lateral hinge line between said shoulder fold and said arm wrap.

18. An arm restraint according to claim 15, and comprising a neck fold formed with said shoulder fold and adapted for residing adjacent a neck of the patient.

19. An arm restraint according to claim 18, and comprising a lateral hinge line between said neck fold and said shoulder fold.

20. A method for limiting arm movement of a patient, comprising the steps of:
(a) applying a fabric arm wrap to an arm of the patient such that a first end of the wrap resides adjacent an upper arm of the patient and a second end of the wrap resides adjacent the wrist;
(b) adjusting the fit of the arm wrap around the arm of the patient to restrict bending of the arm at the elbow;
(c) applying a fabric shoulder fold over a shoulder of the patient adjacent the upper arm, the shoulder fold being formed with the first end of the arm wrap; and
(d) extending a torso strap secured to the shoulder fold at a first attachment point around an upper torso of the patient, and attaching a free end of the torso strap to the shoulder fold at a second attachment point, the torso strap operating to secure and maintain the arm restraint in position on the arm of the patient.
